(19) United States
(12) Patent Application Publication
(10) Pub. No.: US 2025/0186061 A1
(43) Pub. Date: Jun. 12, 2025

(54) SURGICAL INSTRUMENT

(71) Applicant: KYOCERA Corporation, Kyoto-shi, Kyoto (JP)

(72) Inventor: Akinori MORI, Kyoto-shi, Kyoto (JP)

(21) Appl. No.: 18/845,292

(22) PCT Filed: Mar. 1, 2023
(86) PCT No.: PCT/JP2023/007564

§ 371 Date: Sep. 9, 2024

(30) Foreign Application Priority Data
Mar. 11, 2022 (JP) 2022-038497

(51) Int. Cl. A61B 17/15

(52) U.S. Cl. CPC A61B 17/15

(57) ABSTRACT

To achieve improvement in terms of a burden on an operator when performing position adjustment for determining a target position of treatment in surgery without increasing the size of a surgical instrument. The surgical instrument includes a main body, a mover, and a fixer. The main body has a first male screw on an outer circumferential surface and a second female screw on an inner circumferential surface. The mover has a position indicator indicating a position and a first female screw with which the first male screw is screwed. The fixer has a second male screw that is screwed with the second female screw. The first male screw and the second male screw are in a reverse-thread screw relationship.

### TECHNICAL FIELD

The present disclosure relates to a surgical instrument, and in particular to a surgical instrument used in artificial knee joint replacement surgery.

### BACKGROUND OF INVENTION

A surgical instrument for an artificial knee joint replacement surgery disclosed in Patent Document 1 has been known. Patent Document 1 discloses an orthopaedic surgical instrument system including a surgical reamer, a housing, a locking knob, an attachment frame, and a cutting block. The orthopaedic surgical instrument system is capable of advancing part of the housing into engagement with the surgical reamer when the locking knob is rotated to a second position.

### CITATION LIST

### Patent Literature

Patent Document 1: JP 2019-516488 T

### SUMMARY

A surgical instrument according to an aspect of the present disclosure includes: a main body having a first male screw on an outer circumferential surface of a cylinder and a second female screw on an inner circumferential surface of the cylinder; a mover including a position indicator that indicates a target position and a first female screw screwed with the first male screw; and a fixer including a second male screw screwed with the second female screw, wherein rotation of the mover in a rotation direction of the first male screw is restricted, and the first male screw and the second male screw are in a reverse-thread screw relationship.

### BRIEF DESCRIPTION OF THE DRAWINGS

FIG. 1 is a perspective view illustrating an example of how a surgical instrument according to a first embodiment of the present disclosure is used.

FIG. 2 is a perspective view of the surgical instrument illustrated in FIG. 1.

FIG. 3 is a front view of the surgical instrument illustrated in FIG. 1.

FIG. 4 is a side view of the surgical instrument illustrated in FIG. 1.

FIG. 5 is an exploded perspective view of the surgical instrument illustrated in FIG. 1.

FIG. 6 is a partially cutaway perspective view of a screw structure illustrated in FIG. 1.

FIG. 7 is a cross-sectional view of the screw structure illustrated in FIG. 6.

FIG. 8 is a schematic view of the screw structure illustrated in FIG. 6.

FIG. 9 is a diagram illustrating an initial state of position adjustment for the surgical instrument illustrated in FIG. 1.

FIG. 10 is a diagram illustrating a state after the position adjustment for the surgical instrument illustrated in FIG. 1.

### DESCRIPTION OF EMBODIMENTS

### First Embodiment

A first embodiment of the present disclosure will be described below based on the drawings. An example is described below in which a surgical instrument is a guide instrument that indicates a cutting position at a proximal end of a tibial bone in artificial knee joint replacement surgery. However, the present disclosure is not limited thereto, and the surgical instrument may be a guide instrument for indicating a cutting position on the distal end side of the femoral bone in artificial knee joint surgery. For example, the surgical instrument can be applied to position adjustment for a surgical procedure instrument for artificial hip joint replacement, artificial ankle joint replacement, or the like.

The drawings may schematically illustrate the width, thickness, shape, and the like of each portion in comparison with the embodiments in order to make the description clearer, thus, the width, thickness, shape, and the like of each portion are not intended to limit the present disclosure.

### Overview

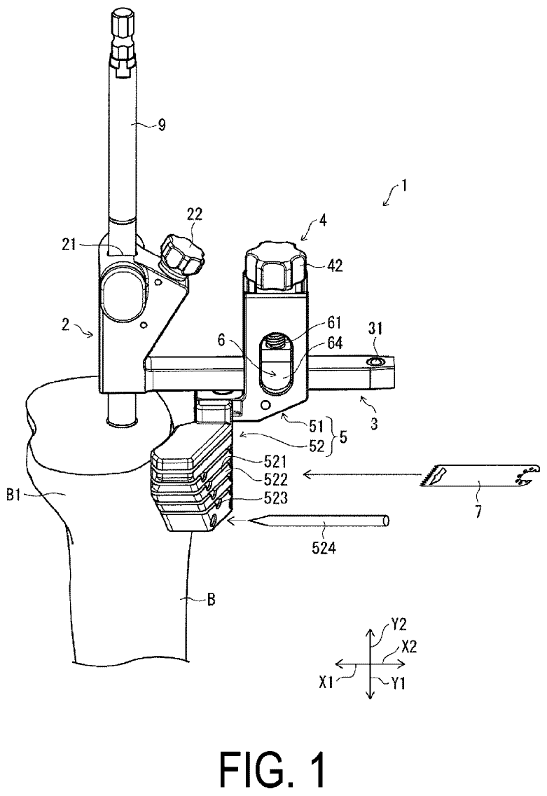

First, in order to facilitate understanding of the surgical instrument 1, an overview of steps of artificial knee joint replacement surgery and an example of how the surgical instrument 1 is used will be briefly described with reference to FIG. 1.

The artificial knee joint replacement surgery is surgery to replace a patient's knee joint with an artificial knee joint. The artificial knee joint includes a tibial implant (not illustrated) to be placed at a proximal end B1 of a tibial bone B of a patient, and a femoral implant (not illustrated) to be engaged with the tibial implant and to be placed at the distal end of a femoral bone (not illustrated).

A user, such as an orthopedic surgeon, performs partial bone resection at each of the proximal end B1 of the tibial bone B and the distal end of the femoral bone of the patient, using a bone resection instrument 7, such as a bone saw, reamer driver, or the like, to form placement surfaces for attachment of the implants. The user attaches the tibial implant and the femoral implant respectively to the placement surfaces of the tibial bone B and the femoral bone, and replaces the patient's knee joint with an artificial knee joint.

The surgical instrument 1 is used, for example, when the bone resection is performed at the proximal end B1 of the tibial bone B to form a placement surface in the above-described steps.

FIG. 1 is a perspective view illustrating an example of how the surgical instrument 1 is used. As illustrated in FIG. 1, a surgical reamer 9 or a rod, which is a separate instrument from the surgical instrument 1, is first inserted into the bone marrow of the tibial bone B. As will be described in more detail below, the surgical instrument 1 is fixedly attached to the surgical reamer 9. The surgical instrument 1 includes a reamer attachment portion 2, a guide member 3, a screw 4, a mover 5, and a fixer 6.

The surgical instrument 1 includes the rod-like guide member 3 extending substantially orthogonally to the surgical reamer 9 when attached to the surgical reamer 9. Here, an insertion direction of the surgical reamer 9 is defined as a Y1 direction (downward direction), and a direction opposite to the Y1 direction is defined as a Y2 direction (upward direction). The directions are indicated by arrows Y1 and Y2 in FIG. 1. Directions parallel to the extending direction of the guide member 3 and substantially orthogonal to the Y1 and the Y2 include a direction toward the surgical reamer 9 referred to as an X1 direction (left direction), and a direction away from the surgical reamer 9 referred to as an X2 direction (right direction). The directions are indicated by arrows X1 and X2 in FIG. 1. The vertical direction may be referred to as a Y direction, and the horizontal direction may be referred to as an X direction.

The surgical instrument 1 described in the present embodiment is for the left leg. One for the right leg has a shape with the reversed relationship between a first portion 52A (described below) and a second portion 52B (described below) with respect to the mover 5.

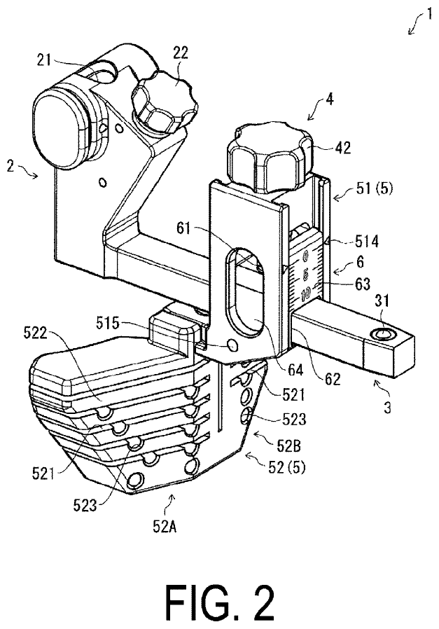

The fixer 6 includes an insertion hole 62 (see FIG. 2) in which the guide member 3 is inserted. The fixer 6 is attached so as to be movable in the X direction along the guide member 3, when the guide member 3 is inserted in the insertion hole 62.

The mover 5 includes a position indicator 52 indicating a cutting position of the tibial bone B. The mover 5 is attached to the fixer 6 using the screw 4. As will be described in detail below, the user can finely adjust the height position of the position indicator 52 by rotating the screw 4 to move the mover 5 in the Y direction. Here, the height position is a position in the Y direction, that is, in the vertical direction. By adjusting the height position, the user can move the position indicator 52 to an appropriate position with respect to the bone resection target portion of the tibial bone B.

Accordingly, the user such as an orthopedic surgeon can perform the bone resection of the tibial bone B at a desired position, with the bone resection instrument 7 inserted in one slit 521 (to be described below) of the position indicator 52.

### Configuration of Surgical Instrument

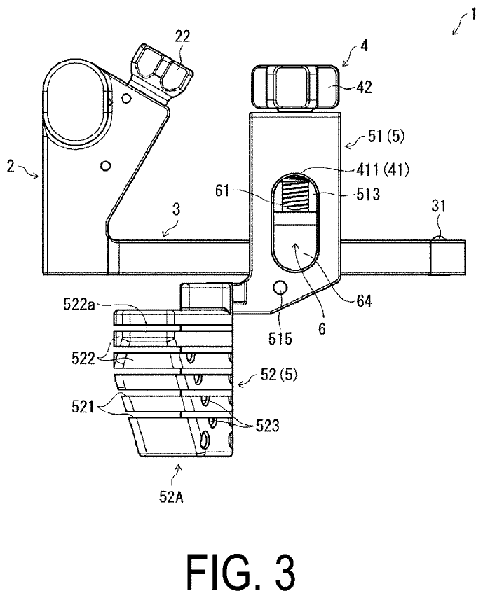

The configuration of the surgical instrument I will be described in detail with reference to FIG. 2 to FIG. 5. FIGS. 2, 3, 4 and 5 are respectively example perspective view, front view, side view, and exploded perspective view of the surgical instrument 1.

As described above, the surgical instrument 1 includes the reamer attachment portion 2, the guide member 3, the screw 4, the mover 5, and the fixer 6.

As illustrated in FIGS. 2 to 5, the reamer attachment portion 2 includes an insertion hole 21 and a screw 22.

The insertion hole 21 is formed over the entire length of the reamer attachment portion 2 in the Y direction. The user inserts the surgical reamer 9, inserted into the bone marrow of the tibial bone B, into the insertion hole 21 from the lower opening of the insertion hole 21 and fixes the surgical reamer 9 using the screw 22. Thus, the user can attach the surgical instrument 1 to the surgical reamer 9.

The user may roughly adjust the height position of the surgical instrument 1 so that the position indicator 52 is located at an appropriate position with respect to the bone resection target portion of the tibial bone B.

The guide member 3 protrudes from a lower portion of the reamer attachment portion 2 and is formed in a rod shape extending in the X direction, that is, in a direction substantially orthogonal to the surgical reamer 9. As described above, the guide member 3 is inserted into the insertion hole 62 of the fixer 6 and is held out by a ball plunger 31. Thus, the fixer 6 is attached to be movable in the X direction along the guide member 3.

The screw 4 is a screw for finely adjusting the height position of the mover 5, and includes a main body 41 and an operation inputter 42 for operating the screw 4. The operation inputter 42 is provided at one end of the main body 41, and is formed to have a larger diameter than the main body 41. In the present embodiment, the operation inputter 42 is provided at a Y2 side end of the main body 41. The user can rotate the main body 41 by rotating the operation inputter 42, and can finely adjust the height position of the mover 5 by rotating the operation inputter 42.

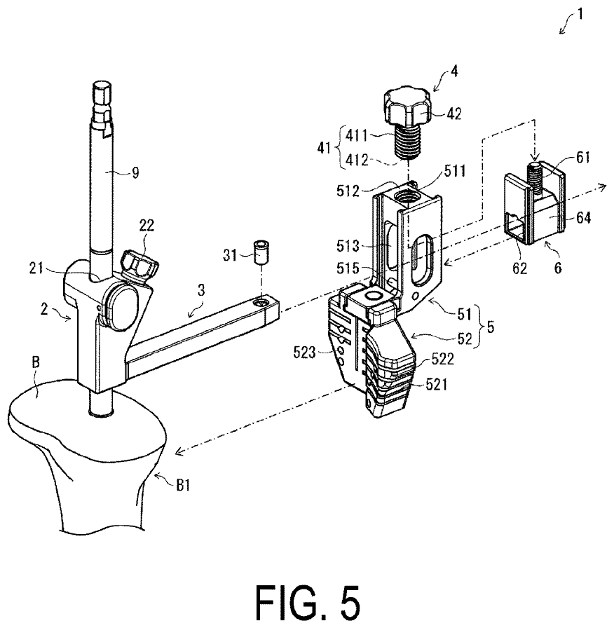

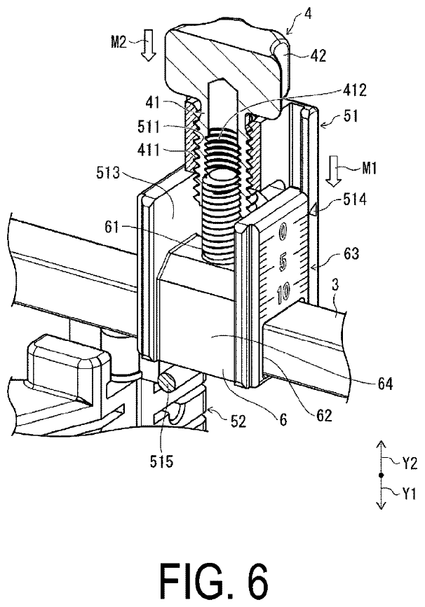

The main body 41 is a shaft of the screw 4 extending in the Y direction and is formed in a cylindrical shape. A first male screw 411 (see FIG. 5) is formed on the outer circumferential surface of the main body 41, and a second female screw 412 (see FIG. 5) is formed on the inner circumferential surface of the main body 41. As illustrated in FIG. 6 described below, the main body 41 has a cylindrical space extending into the main body 41 from the Y1 side end of the main body 41 toward the Y2 side. The second female screw 412 is formed from the Y1 side end of the inner circumferential surface of the cylindrical space to a predetermined position. The cylindrical space of the main body 41 extends to the inside of the operation inputter 42, and has a tapered shape distal end on the Y2 side.

The first male screw 411 and the second female screw 412 are disposed so as to be coaxial with a first female screw 511 (see FIG. 5) of the mover 5 and a second male screw 61 (see FIG. 5) of the fixer 6, which will be described below, in the Y direction.

The second male screw 61 is formed in the fixer 6, and thus is configured not to be rotationally moved or vertically moved.

The screw 4 is joined to the mover 5 when the first male screw 411 is screwed with the first female screw 511, and is joined to the fixer 6 when the second female screw 412 is screwed with the second male screw 61.

As will be described in detail below, the first male screw 411 and the second male screw 61 are configured to be reverse-thread screws. For example, when the first male screw 411 is a left-handed screw, the second male screw 61 is configured to be a right-handed screw. Conversely, when the first male screw 411 is a right-handed screw, the second male screw 61 is configured to be a left-handed screw.

Here, the right-handed screw is a screw threaded in such a manner that the screw is tightened upon being rotated clockwise and is loosened upon being rotated counterclockwise. In other words, the right-handed screw is a screw that advances in a direction to be screwed into the female screw when the male screw is rotated clockwise, and advances in a direction to be removed from the female screw when the male screw is rotated counterclockwise.

On the other hand, the left-handed screw is a screw threaded oppositely compared with the right-handed screw, in such a manner that the screw is tightened upon being rotated counterclockwise and is loosened upon being rotated clockwise. In other words, the left-handed screw is a screw that advances in a direction to be screwed into the female screw when the male screw is rotated counterclockwise, and advances in a direction to be removed from the female screw when the male screw is rotated clockwise.

Since the first male screw 411 and the second male screw 61 are reverse-thread screws, when the user rotates the operation inputter 42 in a direction in which the first male screw 411 is loosened, the mover 5 moves downward so as to be separated from the main body 41 of the screw 4. When the user performs such rotation, the second female screw 412 also rotates and the second male screw 61 of the fixer 6 is screwed into the second female screw 412, whereby the screw 4 moves downward to approach the fixer 6.

In this process, since the mover 5 is attached to the fixer 6, when the operation inputter 42 is rotated, the mover 5 moves downward without rotating together with the screw 4.

Thus, the mover 5 can move by a distance (M1+M2) that is a sum of a downward moving distance (M1) of the mover 5 itself and a downward moving distance (M2) of the screw 4. That is, in a case of a screw structure obtained as a combination of two screws in a reverse-thread screw relationship, i.e., the first male screw 411 and the second male screw 61, an amount of movement with respect to the rotation angle can be made large as compared with a case of implementing the movement with one screw.

As a result, the user can largely move the mover 5 by rotating the screw 4 by a small angle, meaning that the burden on the user associated with the position adjustment can be reduced.

Details of the fine adjustment of the height position of the mover 5 using the screw 4 will be described below.

The mover 5 includes a holder 51 and the position indicator 52 that indicates a bone resection position of the tibial bone B.

The holder 51 includes the first female screw 511, a through hole 512, an accommodation space 513, a mark 514, and a stopper 515.

The first female screw 511 is formed, for example, in the inner circumferential surface of the through hole 512 formed in the upper portion of the holder 51. The first female screw 511 is screwed with the first male screw 411 inserted into the through hole 512 from above the through hole 512.

The accommodation space 513 is formed below the through hole 512, for example, and accommodates the fixer 6 in a state where the guide member 3 is inserted in the fixer 6.

The mover 5 is attached to the fixer 6 via the screw 4 in a state where the fixer 6 is accommodated in the accommodation space 513. Therefore, the user can adjust the distance in the X direction between the position indicator 52 of the mover 5 and the tibial bone B by moving the fixer 6 in the X direction along the guide member 3. Specifically, when the fixer 6 is moved in the X1 direction, the position indicator 52 can be brought closer to the tibial bone B, and when the fixer 6 is moved in the X2 direction, the position indicator 52 can be moved away from the tibial bone B.

The mark 514 is, for example, a triangular mark displayed on one surface of the holder 51. The marks 514 are displayed, for example, on both sides of a scale 63, which will be described below, of the fixer 6 so as to indicate a numerical value or a graduation of the scale 63. Thus, the user can visually recognize the distance of the position indicator 52 from a reference plane P0 for bone resection described below.

The mark 514 may be, for example, a linear or diamond-shaped mark, and is not limited to a triangle. The mark 514 may be displayed only on one side of the scale 63. The details of the mark 514 and the scale 63 will be described below.

The stopper 515 limits the upward movement range of the mover 5. This reduces, for example, damage to the surgical instrument 1, as a result of the position indicator 52 colliding with the guide member 3 due to excessive upward movement of the mover 5. For example, when the mark 514 indicates a predetermined value on the scale 63, the stopper 515 may limit the movement range so that the mover 5 does not move further upward, and the stopper 515 may be disposed at such a position.

The position indicator 52 indicates the cutting position at the proximal end B1 of the tibial bone B. When the tibial bone B is resected, the bone resection instrument 7 is inserted into the position indicator 52, and the position indicator 52 guides the bone resection instrument 7 to the cutting position of the tibial bone B.

The position indicator 52 is detachably coupled to the holder 51. The position indicator 52 includes the first portion 52A and the second portion 52B, each including the slit 521, a guide plate 522, a pin insertion hole 523, and a fixing pin 524.

The first portion 52A is a portion located on the left side (medial, the center side of the body), and the second portion 52B is a portion located on the right side (lateral, the outer side of the body), as viewed in a direction facing the bone resection target portion of the tibial bone B. The first portion 52A and the second portion 52B are disposed while being inclined with each other so as to cover the bone resection target portion of the tibial bone B.

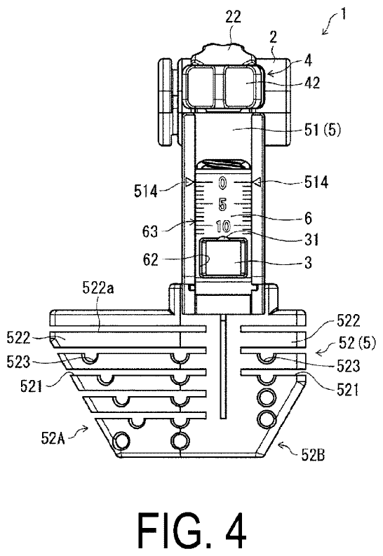

As illustrated in FIG. 4, the first portion 52A and the second portion 52B are not bilaterally symmetrical, and the first portion 52A is configured to have a larger lateral width than the second portion 52B. This is because the surgical instrument 1 is for the left leg, as described above. The first portion 52A and the second portion 52B are laterally asymmetrical as described above, because the surgical procedure involves limitation in terms of the direction of entrance of the bone resection instrument. Specifically, a space exists on the inner side but not on the outer side during the surgery. In contrast to the left leg, for the right leg, a configuration is adopted in which the lateral width of the portion located on the right side is larger than that of the portion located on the left side as viewed in the direction facing the bone resection target portion of the tibial bone B.

The first portion 52A may have the same height in the Y direction as the second portion 52B.

The guide plate 522 is a plate-like member formed to be substantially parallel to the X direction. The position indicator 52 includes a plurality of guide plates 522 arranged to be spaced apart from each other.

The guide plates 522 may be disposed to be spaced apart from each other so as to have an interval corresponding to the height of various implants and the like disposed in the artificial joint. With this configuration, the burden on the user associated with the position adjustment can be further reduced.

The slit 521 is formed in the X direction in a penetrating manner. The position indicator 52 is divided into the plurality of guide plates 522 by the slits 521 in a portion other than boundary portions for the first portion 52A and the second portion 52B.

The slit 521 is formed so as to have a higher height in the Y direction than the bone resection instrument 7 to enable insertion of the bone resection instrument 7.

The slit 521 indicates the cutting position of the tibial bone B. Specifically, the cutting position of the tibial bone B is an upper surface 522a of the guide plate 522, which is a boundary between the slit 521 and the guide plate 522. Since the position indicator 52 includes the plurality of slits 521, a plurality of cutting positions can be indicated through single alignment.

The pin insertion hole 523 is a through hole for inserting the fixing pin 524 for fixing the position indicator 52 to the tibial bone B. After the adjustment of the height position is completed, the position indicator 52 is fixed to the tibial bone B by the fixing pin 524 inserted into the pin insertion hole 523.

After the position indicator 52 is fixed to the tibial bone B, the surgical instrument 1 and the surgical reamer 9 other than the position indicator 52 are removed from the tibial bone B. The user inserts the bone resection instrument 7 into the slit 521 of the position indicator 52, slides the inserted bone resection instrument 7 along the upper surface 522a of the guide plate 522 toward the tibial bone B side, and performs bone resection at a desired position.

In the example described above, the example in which the position indicator 52 includes the plurality of slits 521 is described, but the position indicator 52 is not limited thereto, and may include one slit 521. In this case, the position indicator 52 may include a pair of the guide plates 522 with the slit 521 interposed therebetween, or may include one guide plate 522 without including the slit 521. In this case, the user moves the bone resection instrument 7 along the upper surface 522a of the guide plate 522 to perform the bone resection of the tibial bone B.

The fixer 6 includes the second male screw 61, the insertion hole 62, the scale 63, and a base 64.

The second male screw 61 protrudes upward from the base 64 of the fixer 6 and is screwed with the second female screw 412 formed on the inner circumferential surface of the screw 4 as described above. The second male screw 61 is formed to be in a reverse-thread screw relationship with the first male screw 411.

As described above, the guide member 3 is inserted through the insertion hole 62, and the fixer 6 is attached to the guide member 3. Therefore, the fixer 6 moves in the X direction along the guide member 3 together with the mover 5, but does not move in the Y direction. The fixer 6 is fixed to the surgical reamer 9 inserted into the tibial bone B, via the guide member 3. As a result, the fixer 6 is fixed to the tibial bone B, whereby the cutting position can be accurately presented.

The scale 63 is displayed on one surface of the fixer 6 so as to be visually recognizable by the user, and displays a graduation and/or a numerical value.

The scale 63 is displayed on the surface of the mover 5 on the side corresponding to the side on which the mark 514 is displayed in a state where the fixer 6 is accommodated in the accommodation space 513 of the mover 5. Accordingly, the user can view the scale 63 and the mark 514 at the same time, and thus can visually recognize the graduation or the numerical value on the scale 63 indicated by the mark 514.

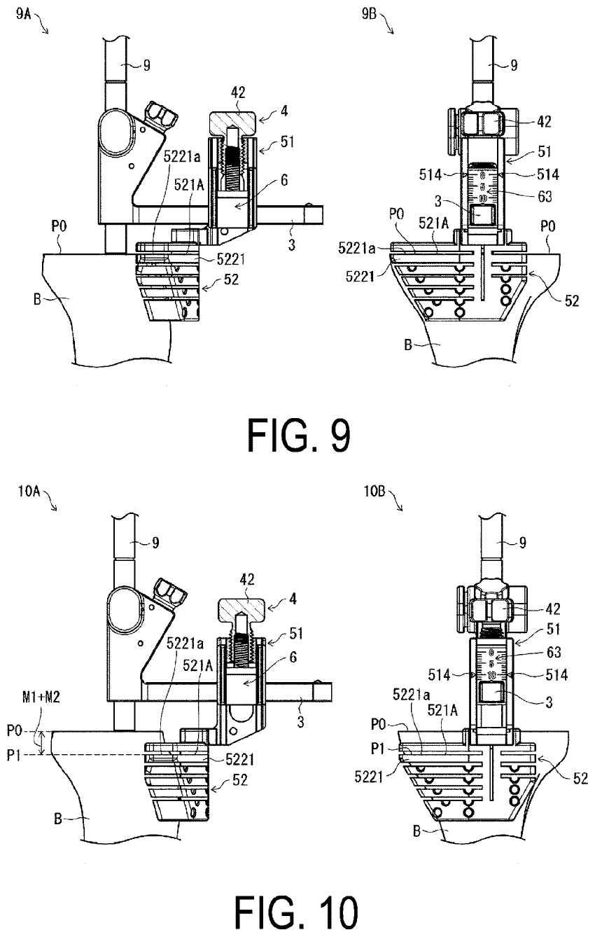

The mark 514 indicates a distance in the Y direction from the reference plane P0 (see FIG. 9) for bone resection described below, with the graduation at the reference plane P0 being 0. More specifically, since the scale 63 is displayed on the fixer 6, the scale 63 does not move in the Y direction, but the mark 514 of the mover 5 moves when the mover 5 moves. Accordingly, the numerical value or the like indicated by the mark 514 indicates the distance of the mover 5 from the reference plane P0 in the Y direction.

### Screw Structure

Movement Amount with Respect to Rotation Angle

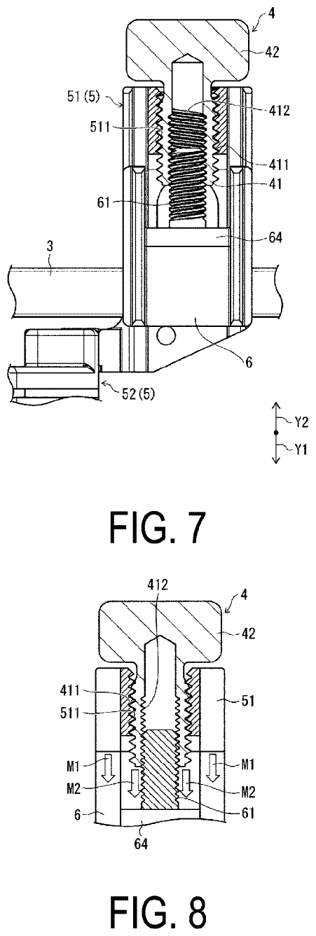

Fine adjustment of the height position of the position indicator 52 using the screw 4 will be described in detail with reference to FIGS. 6 to 8. FIG. 6 is a partially cutaway perspective view of a screw structure, FIG. 7 is a cross-sectional view of the screw structure, and FIG. 8 is an example of a schematic view of the screw structure.

As illustrated in FIGS. 6 to 8, a fine adjustment mechanism for the height position of the position indicator 52 is obtained by the screw structure as a combination of the two screws in a reverse-thread screw relationship, that is, the first male screw 411 and the second male screw 61.

As described above, the first male screw 411, the second female screw 412, the second male screw 61, and the first female screw 511 are configured to be coaxial with each other, the first male screw 411 is screwed with the first female screw 511, and the second male screw 61 is screwed with the second female screw 412.

The first male screw 411 and the second male screw 61 are configured to be reverse-thread screws. The second male screw 61 is formed in the fixer 6, and thus is configured not to be rotationally moved or vertically moved.

Hereinafter, an example will be described in which the first male screw 411 is a left-handed screw, the second male screw 61 is a right-handed screw, and the position indicator 52 is moved downward.

When the user rotates the operation inputter 42 clockwise as viewed from the side opposite to the side where the fixer 6 is screwed, that is, from above the screw 4, the main body 41 rotates clockwise.

When the user rotates the main body 41 clockwise, the main body 41 is moved in a direction (Y1) approaching the fixer 6 by the second male screw 61, and the mover 5 is moved in a direction (Y1) away from the fixer 6 by the first male screw 411.

As a result, the mover 5 moves from the position before the movement by the amount (M2+M1) that is the sum of the amount (M1) of movement caused by the first male screw 411 and the amount (M2) of movement caused by the second male screw 61. Therefore, as compared with the case where the movement is implemented by one screw, the movement amount at the same rotation angle can be increased.

That is, in a case of a screw structure obtained as a combination of two screws in a reverse-thread screw relationship, i.e., the first male screw 411 and the second male screw 61, an amount of movement with respect to the rotation angle can be made large as compared with a case of implementing the movement with one screw.

As a result, the user can largely move the mover 5 by rotating the screw 4 by a small angle, meaning that the burden on the user associated with the position adjustment during the surgery can be reduced.

Normally, when the user rotates the screw clockwise, the male screw is screwed into the female screw. That is, the male screw advances in a direction toward the female screw. In the first embodiment, the first male screw 411 is a left-handed screw. Accordingly, when the user rotates the main body 41 clockwise, the mover 5 moves away from the main body 41. That is, since the mover 5 moves in a direction opposite to the main body 41, the mover 5 can be moved so as to fit the user's natural perception.

However, not limited to this configuration, the first male screw 411 may be a right-handed screw, and the second male screw 61 may be a left-handed screw.

Since the second female screw 412 to be screwed with the second male screw 61 is formed on the inner circumferential surface of the first male screw 411, the second male screw 61 has a smaller diameter than the first male screw 411, and provides a small movement amount (lead) in response to a single rotation, as compared with the first male screw 411. Accordingly, the mover 5 can be moved by using the first male screw 411 and the second male screw 61 in a balanced manner.

### Size of Screw Structure

When the movement amount that is the same as the sum of the amounts of movement caused by the first male screw 411 and the second male screw 61 is to be achieved by a single screw, the single screw needs to have a larger diameter and a longer shaft length. The larger diameter and the longer shaft length of the screw lead to an increase in the size of components including the screw, resulting in obstruction the user's field of view. For this reason, a problem may occur in artificial knee joint replacement surgery which requires precise treatment.

On the other hand, with the screw structure according to the first embodiment, the movement amount can be increased without increasing the size of the screw, and the surgical instrument 1 can be made compact.

### Multi-Start Screw

In the screw structure according to the first embodiment, the first male screw 411 and/or the second male screw 61 may be a multi-start screw such as a double-start screw for example. Thereby, the amount of movement with respect to the rotation angle can be further increased. When it is desired to finely set the amount of movement per rotation, only one of the screws may be a multi-start screw.

### Position Adjustment

An example in which the bone resection instrument 7 is inserted into a slit 521A and the bone resection is performed at a position lower than the position of the reference plane P0 by 10 mm will be described below with reference to FIGS. 9 and 10.

FIG. 9 is a diagram illustrating an example of an initial state of position adjustment for the surgical instrument 1, in which a diagram indicated by reference numeral 9A is a front view, and a diagram indicated by reference numeral 9B is a side view. FIG. 10 is a diagram illustrating an example of an after the position adjustment for the surgical instrument 1, in which a diagram indicated by reference numeral 10A is a front view, and a diagram indicated by reference numeral 10B is a side view.

The user first determines the reference plane P0 for the bone resection. The reference plane P0 for the bone resection may be, for example, a plane including one point serving as a reference for the bone resection, but is not particularly limited.

As illustrated in FIG. 9, first, the user rotates the screw 4 to adjust the graduation indicated by the mark 514 to 0. In this state, the user loosens the screw 22 to adjust the position of the reference plane P0 to coincide with the height of an upper surface 5221a of a guide plate 5221 adjacent to the slit 521A. Alternatively, the user may adjust the height position by loosening the screw 22 so that the graduation indicated by the mark 514 becomes 0 in a state where the height position of the reference plane P0 and the height position of the upper surface 5221a of the guide plate 5221 adjacent to the slit 521A coincide with each other.

After adjusting the height position, the user tightens the screw 22 again to fix the surgical instrument 1, thereby securing the reference plane P0 with the graduation being 0. The height position adjustment thereafter is performed with reference to the reference plane P0.

If it is desired to adjust the end surface of the tibial bone B at the position of the reference plane P0, the user inserts the bone resection instrument 7 into the slit 521A, slides the bone resection instrument 7 along the upper surface 5221a of the guide plate 5221, and performs the bone resection for the tibial bone B.

As illustrated in FIG. 10, the user adjusts the height position of the mover 5 by rotating the screw 4 so that the graduation indicated by the mark 514 becomes 10. The position where the graduation is 10 is a position where a position P1 lower than the position of the reference plane P0 by 10 mm coincides with the height position of the upper surface 5221a of the guide plate 5221.

At the position where the graduation is 10, the user inserts the bone resection instrument 7 into the slit 521A, slides the bone resection instrument 7 along the upper surface 5221a of the guide plate 5221, and performs the bone resection of the tibial bone B.

### Second Embodiment

Another embodiment of the present disclosure will be described below. For the sake of convenience of description, members having the same functions as those of the members described in the above-described embodiment are denoted by the same reference signs, and description thereof is not repeated.

The mover 5 according to the second embodiment is different from the first embodiment in that the mover 5 according to the first embodiment includes the holder 51 and the position indicator 52, but the mover 5 according to the second embodiment includes the holder 51 and a treater 53 (not illustrated) including the bone resection instrument 7.

The height position of the treater 53 can be finely adjusted by rotating the screw 4. Therefore, the position adjustment of the bone resection instrument 7 can be directly performed, whereby the efficiency of surgery can be improved.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, a person skilled in the art can easily make various variations or modifications based on the present disclosure. Note that these variations or modifications are included within the scope of the present disclosure.

### REFERENCE SIGNS

1 Surgical instrument
5 Mover
6 Fixer
41 Main body
52 Position indicator
53 Treater
61 Second male screw
411 First male screw
412 Second female screw
511 First female screw
521 Slit

1. A surgical instrument, comprising:
a main body comprising a first male screw on an outer circumferential surface of a cylinder and a second female screw on an inner circumferential surface of the cylinder;
a mover comprising a position indicator that indicates a target position and a first female screw screwed with the first male screw; and
a fixer comprising a second male screw screwed with the second female screw, wherein
rotation of the mover in a rotation direction of the first male screw is restricted, and the first male screw and the second male screw are in a reverse-thread screw relationship.

2. The surgical instrument according to claim 1, wherein the mover is movably disposed on an outer surface of the fixer.

3. The surgical instrument according to claim 1, wherein the position indicator is a guide that indicates a cutting position of a bone as the target position.

4. The surgical instrument according to claim 3, wherein the cutting position of the bone is a cutting position at a proximal end of a tibial bone.

5. The surgical instrument according to claim 3, wherein the guide comprises at least one slit.

6. The surgical instrument according to claim 3, wherein the fixer is fixable to a separate instrument fixed to the bone.

7. The surgical instrument according to claim 1, wherein a screw thread of the first male screw is provided to make the mover move away from the main body when the main body is rotated clockwise as viewed from a side opposite to a side where the fixer is screwed.

8. The surgical instrument according to claim 1, wherein an amount of advancement as a result of rotating the second male screw once is smaller than an amount of advancement as a result of rotating the first male screw once.

9. The surgical instrument according to claim 1 further comprising an operation inputter provided at one end portion of the main body and being capable of rotating the main body, wherein
a diameter of the operation inputter is larger than a diameter of the main body.

10. The surgical instrument according to claim 1, wherein the first male screw and/or the second male screw is a multi-start screw.
